SEQUENCE LISTING (16 sequences)

SEQ ID NO: 1 - DNA, 462 bp, Methanococcoides burtonii
atgaaatttggattagtacgtaggggttcctctgatgtttcacgctgggatccgtttgatgagatcaggcagactcaggaacacctcaatcagttattaagggaagtctctcctttcgggggattgttcgaaggtaaatcaagggcacctttgatggacatcaaggaagaggataataacgttatcgttacgactgatcttcctggaattgataaagaggatgttgagatcagtgtgaataataatattcttgagatccatgcagagttcaagaaggaaagtgagtctgaaaaggaaggttacgtacaaaaagagcgcacctatagtagcttctcaagatctgctgttcttccctccgtggtttcggatgaaggtgtaaaagcaaagttggaagccggtgtattgaccataacgcttccaaagacaaaagctgaagaaaaaacaaagatcaagatcgagtga

SEQ ID NO: 2 - protein, 153 aa, Methanococcoides burtonii
MKFGLVRRGSSDVSRWDPFDEIRQTQEHLNQLLREVSPFGGLFEGKSRAPLMDIKEEDNNVIVTTDLPGIDKEDVEISVNNNILEIHAEFKKESESEKEGYVQKERTYSSFSRSAVLPSVVSDEGVKAKLEAGVLTITLPKTKAEEKTKIKIE

SEQ ID NO: 3 - DNA, 1629 bp, Methanococcoides burtonii
ttacatcattggaggcattccgcccatgccgccgtctggcattggtggtgctctggatgatgcaatgatatcgtcgatcctgaggatcattactgctgcttctgtgccggagttgatcgcctgggtctttaccctgagtggctcaacaacgcctgcttcccacatgtcaatgacagtgcctgtgtagacgttaagaccagcggtctttatgcctttctcatggtgtgcacgaagttctactagcatgtctatggggtcaagacctgcgttttctgcaagcgttcttggaactacctcaagtgcttctgcgaatgctttgactgcaagctgctctcttccgctaagggttgatgcatactcgttgagtctgagcgcaacctctacttcaggtgctccgccgccagcaacaagctcttcatcttcgatagctacagcgactacacggagtgcatcgttgagtgctctctcgatgttgtcgataacatgctctgtaccgccgcgaaggagaatggatactgactttggattgacacagcctgtgatgaaggtcatgctgtcgccgccgatcttcttctcttcgacaagttctgctgcaccaaggtcttctgctgtcatctcttcgatgttggtgatgagcttagcactggtggagcgtacaagtttctccatgtcgctcttcttgacacgccttactgcaaagataccttcctttgcaaggtagtgctgtgccatatcatcgatacctttctggcagaatacaacgtttgcaccggtctttgtaatactggtgacaaggctcttgagcattgattcttcctggtcgaggaaggactggagctgttcaggagacgtgatagagatctcagcgtcaacctctgtttccttgagctcgatagcactgttgaggagtgcgatccttgcgccttctaccttctttggcatgttggtgtgtaccctttctttatcaaggatcataccttcgataagctcagagtcatcgatacgtccgccgaccttcttctcgaccttgacattctcgatatcgacagtgtttccattgtccctgtcaacgatgcttatgatagcgtcaacagcgatctttgaaagtatgtcctttgttgcttctgcgccctttccggtcattgctgtgtcggaaatgctgataagcatgtccttgttgtcgatggttaccttctttgcaaggctcttgaggatctctcctgcctttactgaagccattctgtaaccagctgcgatgattgttgggtgtatgtcctgctcgatcatctcttccgctttcttgaggagctcaccggtaatgacagcggcggtagttgttccgtctccgacttcatcgtcctgtgttttagcgacctctacgatcatctttgctgctgggtgctcgatatccatttccttaaggatagttgcgccatcgtttgtgataactacatctccaagggagtcaacaagcatcttgtccataccttttggaccaagtgttgttcttactgcctcagcgactgcttttgcagccatgatgttgttgctctgagcatctctgcctcttgttctctggttaccttctcttaaaatgaagataggttgtcctgacatctgtcctgccat

SEQ ID NO: 4 - protein, 542 aa, Methanococcoides burtonii
MAGQMSGQPIFILREGNQRTRGRDAQSNNIMAAKAVAEAVRTTLGPKGMDKMLVDSLGDVVITNDGATILKEMDIEHPAAKMIVEVAKTQDDEVGDGTTTAAVITGELLKKAEEMIEQDIHPTIIAAGYRMASVKAGEILKSLAKKVTIDNKDMLISISDTAMTGKGAEATKDILSKIAVDAIISIVDRDNGNTVDIENVKVEKKVGGRIDDSELIEGMILDKERVHTNMPKKVEGARIALLNSAIELKETEVDAEISITSPEQLQSFLDQEESMLKSLVTSITKTGANVVFCQKGIDDMAQHYLAKEGIFAVRRVKKSDMEKLVRSTSAKLITNIEEMTAEDLGAAELVEEKKIGGDSMTFITGCVNPKSVSILLRGGTEHVIDNIERALNDALRVVAVAIEDEELVAGGGAPEVEVALRLNEYASTLSGREQLAVKAFAEALEVVPRTLAENAGLDPIDMLVELRAHHEKGIKTAGLNVYTGTVIDMWEAGVVEPLRVKTQAINSGTEAAVMILRIDDIIASSRAPPMPDGGMGGMPPMM

SEQ ID NO: 5 - DNA, 444 bp, Methanococcus jannaschii
ttattcagctttttcttcattttcttcctcttctgctttttcttcttcagatgtttgttgagcttctgcaattaaatcctctatttttgcatacaattcggcaattgcttgctctaagactaatctgaatgtcaatagctttttaatttcatcttcaatgtatttcaatgcctcctcatactctaactcagctgaaatattctgtccaactgaaacaacaaccttatccatcttttcaactttcatctctacttgagcaatacttccaacaggaactaagacagttttcccctctcccaatgtttttaagctctttaatgttgctaatgactgtctcaatgttgctattgttgcgtctaatcttccaatttcagctctcaaaccttcaatttgagctatgtatgctctaactgcttcatttatgtctatgacttcatttaccat

SEQ ID NO: 6 - protein, 147 aa, Methanococcus jannaschii
MVNEVIDINEAVRAYIAQIEGLRAEIGRLDATIATLRQSLATLKSLKTLGEGKTVLVPVGSIAQVEMKVEKMDKVVVSVGQNISAELEYEEALKYIEDEIKKLLTFRLVLEQAIAELYAKIEDLIAEAQQTSEEEKAEEEENEEKAE

SEQ ID NO: 7 - DNA, 504 bp, Pyrococcus furiosus
ctattcaactttaacttcgaatccttcactctccttctttgttgggtgcttctttggaactctgatctcaagcactccgttgttgtacttggcctttgccttctctggaataacttcttctggaagcctgatggctcttctataccctgtaaagtatctctctattctcactgctccttctctttctaattctttctccctcttaactgtggcctcaatgtatactgtatcctctgtaaccctcactttgatgtcttcttttctcactcctggaagctctgccgtgattacaaactcatctccgttgtcaaagatatcaacgaatggctctctccagacttctcctactctctcctcatacattgctggctcgctccaccttctgtaagtccagagccttggcctgctgaagaattcatcgaacattgcatcaatttcctcttgtatttcccttattaggtcgaatggatcccatatgtcccatcttcttattctcctcaccat

SEQ ID NO: 8 - protein, 167 aa, Pyrococcus furiosus
MVRRIRRWDIWDPFDLIREIQEEIDAMFDEFFSRPRLWTYRRWSEPAMYEERVGEVWREPFVDIFDNGDEFVITAELPGVRKEDIKVRVTEDTVYIEATVKREKELEREGAVRIERYFTGYRRAIRLPEEVIPEKAKAKYNNGVLEIRVPKKHPTKKESEGFEVKVE

SEQ ID NO: 9 - DNA, 441 bp, Pyrococcus furiosus
tcacttcttaagcttgaagctcattgcttgtttttgttggatctcttgtgccttctttgctagctcagctgctctcttctgaagctcgttgagagcttcctgggtctttcttattgcttcatcgtactctttgatcctctcatccagatatttaattgcatcttcaagcgtcttctcaactgcatatccagaaccaacgctgattattgcgttgttcttatccactatctttcctttaagaaaagacccagcacctataggaactagaatttcaggattctcatcctcaattttcattaggttttccagagtttcctttacagtttgaacctccgcctgtgctaagctgagaagctccaaattttgggccaaaagctgagcttgggcctgtacaacttggtactcataagcaaccttttccaattccttattgttttccat

SEQ ID NO: 10 - protein, 146 aa, Pyrococcus furiosus
MENNKELEKVAYEYQVVQAQAQLLAQNLELLSLAQAEVQTVKETLENLMKIEDENPEILVPIGAGSFLKGKIVDKNNAIISVGSGYAVEKTLEDAIKYLDERIKEYDEAIRKTQEALNELQKRAAELAKKAQEIQQKQAMSFKLKK

SEQ ID NO: 11 - DNA, 522 bp, Pyrococcus furiosus
atgaatcccagggtgtgccaagtggatttaggagctttgtttacaagggagaaggggcctacatttggaggagtcccgagacagttaacgttgagtttggaaaacttctatcccagggtgaggaaggttcttaaatattcctctgaagttagagctgaggtggtgaagatgcaaaacattccaccccaagtccaggcaatgcttgggcaattagagagctaccagcagcaactccaactcgttattcagcagaagcagaaggttcaagctgatttaaatgaagctaaaaaggcccttgaggaaattgaaaagctcactgatgatgctgtaatttacaagaccgttggcacgttgatagttaaaacgacaaaagaaaaagctttacaggaacttaaggagaaagtagaaactcttgaagttaggcttaatgcgctaaacaggcaagagcagaagataaatgaaaagataaaggagctcactcaaaagattcaggcagccctaagacctccaaccgctggatga

SEQ ID NO: 12 - protein, 173 aa, Pyrococcus furiosus
MNPRVCQVDLGALFTREKGPTFGGVPRQLTLSLENFYPRVRKVLKYSSEVRAEVVKMQNIPPQVQAMLGQLESYQQQLQLVIQQKQKVQADLNEAKKALEEIEKLTDDAVIYKTVGTLIVKTTKEKALQELKEKVETLEVRLNALNRQEQKINEKIKELTQKIQAALRPPTAG

SEQ ID NO: 13 - DNA, 1650 bp, Pyrococcus furiosus
atggcccagttagcaggccaacccattctaattttgcctgaaggaacccaaagatacgttggtagagatgcccagagaatgaacattcttgctgctagaattgttgcagagacaataagaacaaccctcggaccaaagggaatggacaagatgctcgttgatagccttggagacatcgtaataacaaacgacggtgcaacaattctcgatgagatggacattcagcacccagcagctaagatgatggttgaggtcgcaaagacccaggacaaggaggccggtgatggaacaacaaccgctgtagtaattgcaggtgagctcctaagaaaggctgaagaattactagaccagaacattcacccaagcataatcatcaaaggttacaccttagcagcacaaaaggctcaagagatcctcgagaacatagccaaagaagtcaagcccgacgatgaggaaattctcctcaaggctgcaatgacatcaattaccggtaaggccgctgaggaggagagggagtacttagccaagcttgcagtagaggcagttaagctagttgcagagaaggaagacggaaagtacaaggttgacatcgacaacatcaagctcgagaagaaggagggtggaagcgtcagagacacccagctcataagaggtgtagttattgacaaggaagtagtccacccaggaatgccaaagagagtcgagaaagctaagattgcactaattaacgatgcacttgaggttaaggagaccgagactgatgccgagataagaattaccagcccagagcaactccaggccttcctcgagcaagaggagagaatgctcagagagatggtcgagaagatcaaggaagtcggagctaacgtagtattcgtccagaagggaattgacgatctagcacagcactacctagccaaatacggaataatggccgtcagaagagtcaagaagagcgacatggagaagctcgccaaggccacaggagctaagatcgtaaccaacattagggacctcacaccagaggacctcggttacgctgagctagtagaagagagaaaggttgctggagagagcatgatattcgtcgagggctgccagaaccccaaggctgtgacaatcctcatcagaggtggaactgagcacgtagtcgatgaggtcgagagagccctagaagatgcaataaaggttgtgaaggacatccttgaagatggaaagatcctagctggcggtggagcaccagaaatcgagttagccattagactcgacgagtacgccaaggaagttggtggcaaggagcagttggcaattgaggcctttgcagaggctctcaaggtcattccaaggacactagcagagaacgctggtctcgacccaatcgagacactcgttaaggtcatcgctgcccacaaggagaagggaccaaccatcggtgtcgatgtatacgaaggcgaaccagcagacatgctagagagaggagtcatcgagccactaagagtcaagaagcaagctatcaagagtgctagcgaggcagcaataatgatcctcagaatcgacgatgtcatcgctgccagcaagctcgagaaagagaaggagaaagaaggtgagaagggaggaggaagcgaggacttcagcagtgatctagactga

SEQ ID NO: 14 - protein, 549 aa, Pyrococcus furiosus
MAQLAGQPILILPEGTQRYVGRDAQRMNILAARIVAETIRTTLGPKGMDKMLVDSLGDIVITNDGATILDEMDIQHPAAKMMVEVAKTQDKEAGDGTTTAVVIAGELLRKAEELLDQNIHPSIIIKGYTLAAQKAQEILENIAKEVKPDDEEILLKAAMTSITGKAAEEEREYLAKLAVEAVKLVAEKEDGKYKVDIDNIKLEKKEGGSVRDTQLIRGVVIDKEVVHPGMPKRVEKAKIALINDALEVKETETDAEIRITSPEQLQAFLEQEERMLREMVEKIKEVGANVVFVQKGIDDLAQHYLAKYGIMAVRRVKKSDMEKLAKATGAKIVTNIRDLTPEDLGYAELVEERKVAGESMIFVEGCQNPKAVTILIRGGTEHVVDEVERALEDAIKVVKDILEDGKILAGGGAPEIELAIRLDEYAKEVGGKEQLAIEAFAEALKVIPRTLAENAGLDPIETLVKVIAAHKEKGPTIGVDVYEGEPADMLERGVIEPLRVKKQAIKSASEAAIMILRIDDVIAASKLEKEKEKEGEKGGGSEDFSSDLD

SEQ ID NO: 15 - DNA, 336 bp, Pyrococcus furiosus
ctaaggagagccttcagtaagctttagtatcgcttccgctaaatctccattagcttcttccaaagccttttttgctgtttcatagtcaacaccagtctgctccattacaagtttaatatcttcttcagatattactagttttactttctcctcttcttctcctccagcaatttgatatatcttttctcccatggcttttatcacggtcacagttggattttttattataatctctctgtcttcgagccttatgataacttcaacaacgttatcaagctgtctcatgtcaagttgcttcatcaattttttgagctgttttgggttcattggcatcat

SEQ ID NO: 16 - protein, 111 aa, Pyrococcus furiosus
MMPMNPKQLKKLMKQLDMRQLDNVVEVIIRLEDREIIIKNPTVTVIKAMGEKIYQIAGGEEEEKVKLVISEEDIKLVMEQTGVDYETAKKALEEANGDLAEAILKLTEGSP

(19) United States
(12) Patent Application Publication
(10) Pub. No.: US 2012/0064560 A1
(43) Pub. Date: Mar. 15, 2012

(54) FOLDING OF RECOMBINANT PROTEINS VIA CO-EXPRESSION OF ARCHAEAL CHAPERONES

(76) Inventors: Harold E. Smith, Bethesda, MD (US); Frank T. Robb, Gaithersburg, MD (US)

(21) Appl. No.: 12/999,185

(22) PCT Filed: Jun. 16, 2009
(86) PCT No.: PCT/US09/47455

§ 371 Date: Sep. 29, 2011

Related U.S. Application Data
(60) Provisional application No. 61/061,759, filed on Jun. 16, 2008.

(51) Int. Cl. C12Q 1/02; C07H 21/04; C12N 1/21

(52) U.S. Cl. 435/29; 435/252.33; 536/23.7

(57) ABSTRACT

The present invention relates to recombinant protein production, and more specifically, to methods for recovery of properly folder bioactive proteins by expressing chaperone genes from extremophilic Archaea, during recombinant protein synthesis in a host cell thereby significantly improving recovery of properly folded bioactive proteins.

### CROSS-REFERENCE TO RELATED APPLICATIONS

This application claims priority to U.S. Provisional Application No. 61/061,759, filed on Jun. 16, 2008, the contents of which are hereby incorporated by reference herein for all purposes.

### BACKGROUND OF THE INVENTION

1. Field of Invention

The present invention relates to recombinant protein production, and more specifically, to methods for recovery of properly folder bioactive proteins by expressing chaperone genes from extremophilic Archaea, during recombinant protein synthesis in a host cell thereby significantly improving recovery of properly folded bioactive proteins.

2. Description of the Related Art

The efficient production of genetically engineered proteins is essential for research and industrial applications. Recombinant DNA technology makes available simple strategies for transferring and efficiently expressing genes of interest in a foreign host cell. Protein production in bacteria, typically Escherichia coli, offers a number of advantages over other systems: ease of transformation and culture growth, a wide range of inducible expression vectors that produce large amounts of protein, and a variety of epitope tags that permit one-step affinity purification. While alternative expression systems are better suited for proteins requiring extensive post-translational modification, the bacterial synthesis of recombinant proteins will remain a preferred mode of production for the foreseeable future. Applications range from the expression and purification of single proteins for biochemical characterization or structural determination, to the expression of entire biosynthetic pathways from heterologous species to produce naturally occurring compounds, to the engineering of novel metabolic programs envisioned by the developing discipline of synthetic biology.

Despite the many advantages of bacterial expression, protein insolubility remains a major stumbling block for recombinant protein production. Data compiled from high-throughput protein expression projects at structural genomics centers demonstrate the magnitude of this problem. As of February 2008, only 41.4% (30,922 of 74,670; see the Protein Structure Initiative at http://sg.pdb.org/target_centers.html) of expressed target proteins produced soluble protein. The results from some species-specific efforts are even more daunting: projects focused on extremophiles (Jenney and Adams 2008) or Plasmodiumfalciparum (Mehlin et al. 2006) generated soluble protein from 20% or less of the expressed targets.

In vitro refolding of purified inclusion bodies is an unpredictable and time-consuming method, with limited success. Typical strategies include manipulation of culture conditions (such as growth medium, temperature, culture density, and/or inducer concentration), alteration of the coding potential of the recombinant gene (including codon optimization, directed evolution, and host expression of rare codon tRNAs), or modification of the affinity epitope (by shifting to a different position within the protein or substitution of a different epitope tag) (Jana and Deb 2005); (Sorensen and Mortensen 2005). Limited success has been reported for each of these approaches, but none has proved the panacea for the problem of protein insolubility.

Protein folding in vivo is promoted by the activities of protein chaperones in all organisms, and the folding pathway in E. coli has been well characterized (Baneyx and Mujacic 2004); (Hoffmann and Rinas 2004). As the nascent polypeptide exits the ribosome, it is bound by either trigger factor or the DnaK/DnaJ complex (Deuerling et al. 1999) (Teter et al. 1999). ATP-dependent folding of the substrate is facilitated by either DnaK/DnaJ in combination with GrpE, or by the GroEL/GroES complex (Ewalt et al. 1997). The failure to fold properly leads to protein aggregation and binding of the small inclusion body proteins IbpA and IbpB (Allen et al. 1992); (Laskowska et al. 1996). Dissociation of protein aggregates is mediated by ClpB as well as the DnaK/DnaJ/GrpE complex (Zolkiewski 1999). If the folded conformation is not attained, the protein either accumulates as inclusion bodies or is degraded through the activity of one or more proteases.

Insolubility of recombinant proteins is likely a consequence of limited folding capacity in the bacterial host. Therefore, another approach to improving solubility has been to increase the expression of one or more E. coli chaperones during protein induction. Chaperones that have been reported to improve folding include one or more combinations of the nine listed above (trigger factor, DnaK, DnaJ, GrpE, GroEL, GroES, ClpB, IbpA, and IbpB) (Thomas and Baneyx 1996); (Nishihara et al. 2000); (Chen et al. 2002); (Han et al. 2004); (de Marco et al. 2007); (Rinas et al. 2007). The response of recombinant protein solubility to different chaperones is idiosyncratic, with specific chaperone combinations required for maximal solubility of different proteins. This phenomenon reflects the observation that different protein substrates are folded preferentially by different chaperone assemblies. It might also indicate that coordinate regulation of the protein folding pathway is required for optimal activity.

Thus, it would be advantageous to develop methods and systems to improve recovery of recombinant properly folded, bioactive proteins that overcome the shortcomings of the prior art.

### SUMMARY OF THE INVENTION

In one aspect, the present invention relates to a mixture comprising isolated chaperones from an extremophilic, such as a hyperthermophilic and/or psychrophilic archaeon for enhancing the folding of expressed native and/or non-native proteins in a bacteria host.

In another aspect, the present invention relates to a mixture of expressed proteins in a bacteria host, wherein the mixture comprises expressed chaperones from a hyperthermophilic and/or psychrophilic archaeon; and expressed native and/or non-native proteins.

In yet another aspect, the present invention relates to a method of enhancing protein folding in a bacteria host, such as e coli, the method comprising:
providing at least one delivery device for expressing a prefoldin (PFD), heat shock protein, chaperonins, and/or nascent polypeptide-associated complex protein (NAC) from a hyperthermophilic and/or psychrophilic archaeon in combination with expression of a native and/or non-native protein in the host bacteria, wherein the prefoldin, heat shock protein and/or NAC is expressed previously, simultaneously or subsequent to the expression of the native or non-native protein in the host.

A still further aspect relates to a method for enhancing protein folding of a native and non-native protein in a bacteria host to provide increased level of properly folded and bioactive proteins, the methods comprising:

introducing into a bacteria host at least one expression vector comprising:
nucleic acid encoding a chaperone selected from the group consisting of prefoldin (PFD), heat shock protein, chaperonins, and/or nascent polypeptide-associated complex protein (NAC) from a hyperthermophilic and/or psychrophilic archaeon and native or non-native protein; and

culturing the bacteria host under conditions sufficient for expression of the proteins.

Another aspect relates a kit comprising an expression vector for expression of native or non-native proteins to provide for increased levels of proper folding in the expressed proteins, wherein the kit comprises a vector including nucleotide sequences for at least one chaperone selected from the group consisting of prefoldin (PFD), heat shock protein, chaperonins, and/or nascent polypeptide-associated complex protein (NAC) from a hyperthermophilic and/or psychrophilic archaeon and also sufficient room for including nucleotide sequences for expression of a native or non-native protein of choice.

Another aspect of the present invention relates to a method to screen for extremophilic chaperones that exhibit folding activity under E. coli growth conditions, the method comprising;
providing a delivery device comprising a nucleotide sequence that encodes for an extremophilic chaperone and an indicator protein, wherein the indicator protein provides for a detectable signal, such as the green fluorescence protein.

A further aspect relates to a delivery device comprising nucleotide sequences encoding chaperones from a hyperthermophilic and/or psychrophilic archaeon, in an amount to enhance the folding of expressed native and non-native proteins in a bacteria host. The delivery device may further include nucleotide sequences encoding for non-native proteins for expression by the bacterial host.

Yet another aspect relates to an assay to screen for extremophilic chaperones that exhibit folding activity under bacteria growth conditions, the method comprising;
a. expressing a testing extremophilic chaperone in combination with the expression of green fluorescent protein; and
b. determining the amount of amount of GFP recovered in the soluble protein fraction.

Other aspects, features and embodiments of the invention will be more fully apparent from the ensuing disclosure and appended claims.

### BRIEF DESCRIPTION OF THE FIGURES

FIG. 1 shows IPTG induction of GFP expression at 37° C. results in the accumulation of misfolded, non-fluorescent protein. Co-expression of functional chaperone facilitates proper folding, which is detectable by increased GFP fluorescence.

FIG. 2 shows the promotion of GFP fluorescence by chaperones. Mean fluorescence signal intensity of GFP is indicated in arbitrary units. Shown are whole cell measurements two hours after co-induction of GFP plus the indicated chaperone. Samples were repeated in triplicate; fluorescence values were all within 25% of the mean. 1, control lacking chaperone; 2, P. furiosus HSP60; 3, P. furiosus PFD; 4, P. furiosus PFD; 5, P. furiosus NAC; 6, M. burtonii HSP60; 7, M. burtonii sHSP; 8, M. jannaschii PFD.

FIG. 3 shows cell extracts of GFP induction. SDS-PAGE of total cell lysates after two hour induction show equal amounts of GFP protein (arrow). 1, control lacking chaperone; 2, P. furiosus HSP60; 3, P. furiosus PFD; 4, P. furiosus PFD; 5, P. furiosus NAC; 6, M. burtonii HSP60; 7, M. burtonii sHSP; 8, M. jannaschii PFD.

FIG. 4 shows soluble extracts of GFP induction. SDS-PAGE of soluble lysates after fractionation by centrifugation. Samples (10 ug each) represent protein from ˜10× the amount of cells shown in FIG. 3. Numbers below indicate the relative amount of GFP by densitometric scan compared to the control. 1, P. furiosus HSP60; 2, P. furiosus PFD; 3, P. furiosus PFD; 4, P. furiosus NAC; 5, M. burtonii HSP60; 6, M. burtonii sHSP; 7, M. jannaschii PFD; 8, control lacking chaperone.

### DETAILED DESCRIPTION OF THE INVENTION

Expression of heterologous genes in Escherichia coli is a routine technology for recombinant protein production, but the predictable recovery of properly folded and bioactive material remains a challenge. Misfolded proteins typically accumulate as insoluble inclusion bodies, and a variety of strategies have been employed in efforts to increase the yield of soluble product. The present invention provides a method using chaperones from extremophiles exhibiting novel folding activities. The green fluorescent protein of Aequorea victoria, which is predominantly insoluble under typical recombinant expression culture conditions, was employed as an in vivo indicator of protein folding activity for chaperone homologs from a variety of extremophiles. For a subset of the chaperones tested, co-expression with GFP promoted an increase in both fluorescence signal intensity as well as the amount of GFP recovered in the soluble protein fraction. This simple and rapid assay provides a tool to screen for extremophilic chaperones that exhibit folding activity under E. coli growth conditions, and shows that increasing the repertoire of heterologous chaperones provides an unexpected and successful solution to the problem of recombinant protein insolubility.

### DEFINITIONS

As used herein, the following terms have the following meanings.

As used herein, the term “heat shock protein” refers to a protein that belong in a class of proteins that were first identified as up-regulated in response to stress, heat. A “heat shock protein” assists in correct protein folding, intracellular protein localization, and other function in the cell to maintain protein structure and function. Stress proteins are grouped into families according to their molecular mass. “Heat shock proteins” for use in the invention include Hsp 60 proteins (chaperonins), which have a molecular weight from about 55-64 kDa, and small Hsp proteins, which have a molecule weight of less than about 35 kDa. Heat shock proteins as broadly defined can encompass chaperones, although not all chaperones are up-regulated in response to heat or other stress.

As used herein, the term “chaperone” refers to a protein that binds to misfolded or unfolded polypeptide chains and affects the subsequent folding processes of the chains. A hallmark of a “chaperone” is the ability to prevent aggregation of normative proteins.

As used herein, the term “chaperonins” refers to a subgroup of “chaperones” that are structurally related and share a stacked ring structure.

As used herein, the term “prefoldin” refers to a chaperone that is found in all Eurkaryotes and Archaea. Prefoldin is typically characterized by a heterohexameric molecular structure that has been referred to as jellyfish-like. Prefoldins have traditionally been grouped into two main evolutionarily related classes: one class that has 140 residues (a prefoldin) and a second class that as 120 residues (β prefoldin). The term “prefoldin” encompasses homologs to α and β prefoldin, e.g., γ prefoldin, that do not associate with either α and β prefoldin to form heteroligomeric complexes.

As used herein, the term “extremophile” refers to an organism that exhibit optimal growth under extreme environment conditions. Extremophiles include acidophiles, alkaliphiles, halophiles, thermophiles (including hyerthermophiles and psychrophile archaeon), metalotolerant organisms, osmophiles, and xerophiles.

As used herein, the terms “nucleic acid” and “polynucleotide” are used synonymously and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages; and peptide nucleic acid backbones and linkages. Other analog nucleic acids include those with positive backbones; non-ionic backbones, and non-ribose backbones. Thus, nucleic acids or polynucleotides may also include modified nucleotides that permit correct read through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

As used herein, the term “a nucleic acid sequence encoding” refers to a nucleic acid which contains sequence information for the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform with codon preference in a specific host cell.

As used herein, the term “gene”, e.g., a prefoldin gene such as γ PFD gene, or a chaperonin gene, refers to a nucleic acid that encodes the chaperone (or heat shock protein), or fragment thereof. Often, such a “gene” is a cDNA sequence that encodes the protein.

As used herein, the term “promoter” or “regulatory element” refers to a region or sequence determinants located upstream or downstream from the start of transcription that direct transcription. As used herein, a promoter includes necessary nucleic acid sequences near the start site of transcription, such as, in the case of a polymerase II type promoter, a TATA element. A promoter also optionally includes distal elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter) and a second nucleic acid sequence, such as a heat shock protein gene or chaperonin gene, wherein the expression control sequence directs transcription of the nucleic acid corresponding to the second sequence.

As used herein, the term “vector” or “delivery device” refers to a replicon, such as a plasmid, phage, cosmid or virus to which another DNA or RNA segment may be attached so as to bring about the replication of the attached segment. Specialized vectors were used herein, containing various promoters, polyadenylation signals, genes for selection, etc.

As used herein, the term “transcriptional and translational control sequences” refer to DNA regulatory sequences, such as promoters, enhancers, polyadenylation signals, terminators, and the like, that provide for the expression of a coding sequence in a host cell.

As used herein, the term “substantial identity” refers to a polynucleotide or polypeptide comprising a sequence that has at least 50% sequence identity to a reference sequence. Alternatively, percent identity can be any integer from 50% to 100%. Exemplary embodiments include at least: 55%, 57%, 60%, 65%, 70%, 75%, 80%, 85%, 90%, 95%, or 99% identity compared to a reference sequence using the programs described herein; preferably BLAST using standard parameters, as described below.

Polypeptides that are “substantially similar” share sequences as noted above except that residue positions that are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

As used herein, the term “isolated”, refers to a nucleic acid or protein that is essentially free of other cellular components with which it is associated in the natural state. It is preferably in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest.

The invention employs various routine recombinant nucleic acid techniques. Generally, the nomenclature and the laboratory procedures in recombinant DNA technology described below are those well known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel et al., eds., 1994-1999 with updates through 2007).

In some embodiments, the chaperone or heat shock protein is from Archaea. There are many Archaea known, including members of the genera Pyrococcus, Thermococcus, Thermoplasma, Thermotoga, Sulfolobus, Halobacterium, and methanogens, e.g., Methanocaldococcus, Methanococcus, Methanothermabacteria; and variohalobacterium. Examples of Archaea include Pyrococcus furiosus; Pyrococcus horikoshii, Sulfolobus solfataricus, Sulfolobus acidocaldarius, Sulfolobus brierleyi, Sulfolobus hakonensis, Sulfolobus metallicus, Sulfolobus shibatae, Aeropyrum pernix; Archaeglobus fulgidus; Thermoplasma acidophilum; Thermoplasma volcanium, Thernotoga maritime, Methanocaldococcus jannaschii; Methanococcoides burtonii, Methanobacterium thermoautotrophicum, Haloferax volcanii, and Halobacterium species NRC-1.

Isolation or generation of heat shock/chaperone polynucleotides can be accomplished by a number of techniques. Cloning and expression of such technique will be addressed in the context of chaperone genes. For instance, oligonucleotide probes based on the sequences disclosed here can be used to identify the desired polynucleotide in a cDNA or genomic DNA library from a desired extremophile species. Probes may be used to hybridize with genomic DNA or cDNA sequences to isolate homologous genes in the same or different plant species.

In some embodiments, the nucleic acids of interest from extremophiles can be amplified from nucleic acid samples using amplification techniques. For instance, PCR may be used to amplify the sequences of the genes directly from mRNA, from cDNA, from genomic DNA or from libraries.

Appropriate primers and probes for identifying a gene from extremophiles, e.g., Archaea, can be generated from sequences available in the art. For a general overview of PCR see PCR Protocols: A Guide to Methods and Applications. (Innis, M, Gelfand, D., Sninsky, J. and White, T., eds.), Academic Press, San Diego (1990).

To express the extremophile sequences, e.g., chaperonin and prefoldin sequences, recombinant DNA vectors suitable for transformation of the organism of interest, e.g., a bacteria, yeast, an archaeal species, a microalgae species, or a microscopic filamentous fungus are prepared. Techniques for transformation are well known and described in the technical and scientific literature. For example, a DNA sequence encoding a prefoldin gene can be combined with transcriptional and other regulatory sequences which will direct the transcription of the sequence from the gene in the intended cells, e.g., bacteria, yeast, and the like. In some embodiments, an expression vector that comprises an expression cassette that comprises the heat shock protein or chaperone gene further comprises a promoter operably linked to the gene. In other embodiments, a promoter and/or other regulatory elements that direct transcription of the gene are endogenous to the microorganism, e.g., yeast, and the expression cassette comprising the heat shock protein or chaperone gene is introduced, e.g., by homologous recombination, such that the heterologous heat shock protein or chaperone gene is operably linked to an endogenous promoter and is expression driven by the endogenous promoter.

Regulatory sequences include promoters, which may be either constitutive or inducible. In some embodiments, a promoter can be used to direct expression of a heat shock protein or a chaperone under the influence of changing environmental conditions. Examples of environmental conditions that may effect transcription by inducible promoters include the presence of a solvent such as ethanol, anaerobic conditions, elevated temperature, or the presence of light. Promoters that are inducible upon exposure to chemicals reagents are also used to express nucleic acids encoding a heat shock protein or chaperone. Other useful inducible regulatory elements include copper-inducible regulatory elements; tetracycline and chlor-tetracycline-inducible regulatory elements; ecdysone inducible regulatory elements and lac operon elements, which are used in combination with a constitutively expressed lac repressor to confer, for example, IPTG-inducible expression. In some embodiments, a promoter that is inducible by the toxic compound, e.g., an ethanol-inducible promoter, is used for the expression of the heterologous extremophile heat shock protein.

A vector comprising a chaperone nucleic acid sequence will typically comprise a marker gene that confers a selectable phenotype on the cell to which it is introduced. Such markers are known, for example, the marker may encode antibiotic resistance, such as resistance to kanamycin, G418, bleomycin, hygromycin, and the like. Further, as used herein the green fluorescent proteins provides not only a signal but also evidence of refolding enhancement.

The chaperone sequences of the invention are expressed recombinantly in an organism of interest, e.g., bacteria, yeast, blue green algae, filamentous fungi, or an archael species. As appreciated by one of skill in the art, expression constructs can be designed based on parameters such as codon usage frequencies of the organism in which the nucleic acid is to be expressed. Codon usage frequencies can be tabulated using known methods (see, e.g., Nakamura et al. Nucl. Acids Res. 28:292, 2000). Codon usage frequency tables are also available in the art e.g., in codon usage databases such as the database developed and maintained by Yasukazu Nakamura at The First Laboratory for Plant Gene Research, Kazusa DNA Research Institute, Japan).

In a preferred embodiment, the chaperones are expressed in bacteria. Particularly useful in the present invention will be cells that are readily adaptable to large-scale culture for production of industrial quantities of proteins. Such organisms are well known in the art of industrial bioprocessing, examples of which may be found in Recombinant Microbes for Industrial and Agricultural Applications, Murooka et al., eds., Marcel Dekker, Inc., New York, N.Y. (1994), and include fermentative bacteria as well as yeast and filamentous fungi. Host cells can includes, e.g., Comamonas sp., Corynebacterium sp., Brevibacterium sp., Rhodococcus sp., Azotobacter sp., Citrobacter sp., Enterobacter sp., Clostridium sp., Klebsiella sp., Salmonella sp., Lactobacillus sp., Aspergillus sp., Saccharomyces sp., Zygosaccharomyces sp., Pichia sp., Kluyveromyces sp., Candida sp., Hansenula sp., Dunaliella sp., Debaryomyces sp., Mucor sp., Torulopsis sp., Methylobacteria sp., Bacillus sp., Escherichia sp., Pseudomonas sp., Serratia sp., Rhizobium sp., and Streptomyces sp., Zymomonas mobilis, acetic acid bacteria, methylotrophic bacteria, Propionibacterium, Acetobacter, Arthrobacter, Ralstonia, Gluconobacter, Propionibacterium, and Rhodococcus.

Cell transformation methods and selectable markers for bacteria, yeast, cyanobacteria, filamentous fungi and the like are well known in the art, and include electroporation, ballistic method, as well as chemical transformation methods.

Conditions for growing bacteria, yeast, or other microorganisms that express a chaperone for the exemplary purposes illustrated above are known in the art. Compounds produced by the modified microorganisms can be harvested using known techniques. For example, compounds that are not miscible in water may be siphoned off from the surface and sequestered in suitable containers.

In typical embodiments, transformed microorganisms that express a heterologous chaperone gene are grown under mass culture conditions for the production of the proteins. The transformed organisms are grown in bioreactors or fermentors that provide an enclosed environment or open environment. In typical embodiments for mass culture, the transformed cells are grown in reactors in quantities of at least about 500 liters, often of at least about 1000 liters or greater, and in some embodiments in quantities of about 1,000,000 liters or more.

The present invention provides methods and systems to expand the folding capacity of recombinant proteins in a bacterial host by the introduction of additional, heterologous chaperone functionality. Chaperones from species within the domain Archaea are particularly suited because various archaeal species have evolved to occupy ecological niches on the limits of biology: high salinity, low pH and extremes of high and low temperature, such as ranges from 15° C. to 2° C. or in the range of 75° C. to 100° C. Each of these environments poses particular challenges to the problem of protein folding. In some instances, homologs of bacterial chaperones that are conserved in Archaea might exhibit folding activities across a wider range of physical conditions than those in E. coli.

The ability of chaperones from a number of archaeal species was examined to show the improvement of in vivo folding of recombinant green fluorescent protein (GFP). Fluorescence requires the proper folding of the protein, providing a rapid and sensitive assay for chaperone activity. A subset of the chaperones tested significantly improved the fluorescence and solubility of GFP under standard culture conditions, thereby demonstrating the utility of this strategy for ameliorating the problem of recombinant protein misfolding and insolubility.

### EXAMPLES

### Materials and Methods

Plasmids: The wild-type gene for green fluorescent protein from the jellyfish Aequorea victoria was amplified from plasmid pPD79.44 (a gift of Andy Fire) by PCR with gene-specific primers that also encoded EcoRI (5′) and NotI (3′) restriction sites. The PCR product was digested with EcoRI and NotI, and cloned into expression vector pET28a (Novagen) digested with the same two restriction enzymes to create pET-GFP. A similar approach was taken for the cloning of archaeal chaperones. Each was amplified from genomic DNA from the respective organism with gene-specific primers that included flanking restrictions sites appropriate for directional cloning into pET expression vectors pET11a (P. furiosus chaperonin; NcoI and BamHI sites), pET19b (P. furiosus prefoldin, prefoldin, and NAC, plus M. jannaschii prefoldin; all NdeI and XhoI), or pETDuet-1 (M. burtonii chaperonin and sHSP; each NdeI and XhoI). For each plasmid, DNA sequencing confirmed that the amplified gene was correct. The expression plasmid for P. furiosus sHSP has been described previously (Laksanalamai et al., 2001).

Protein induction: Plasmids for expression of both GFP and chaperone are under transcriptional control of T7 polymerase (Novagen). E. coli strain BL21(DE3) (Studier and Moffatt 1986) was transformed sequentially with pET-GFP using kanamycin selection, then with one of the pET-chaperone plasmids using ampicillin and kanamycin selection. Cultures were grown in LB medium with antibiotics in a shaking incubator at 37° C. until mid-log phase (OD600=0.6±0.1), then induced with 1 mM IPTG for two hours. To measure GFP fluorescence, cells from an aliquot of culture were pelleted by centrifugation for 5 minutes at 4,000 RCF, then resuspended in water at OD600=0.3±0.02. Whole-cell fluorescence was quantified by a FluoroMax III spectrofluorometer (Horiba) at 397 nm excitation and 504 nm emission. To separate proteins into soluble and insoluble fractions, cells from the remainder of the culture were pelleted by centrifugation and resuspended in extraction buffer (50 mM Tris-HCl, pH 7.4, 150 mM NaCl, 1 mM EDTA, and 10% glycerol). Lysis and fractionation were by one freeze/thaw cycle, treatment with lysozyme and DNAse I, sonification, and centrifugation for 45 minutes at 100,000 RCF. Lysates were subjected to SDS-PAGE on 10-20% gradient gels, then stained with a Coomassie-based dye (Gelcode Blue, Pierce) for protein visualization.

### Results and Discussion

The endogenous protein folding activity of the E. coli host was sought to be enhanced by expressing chaperones from various species of Archaea. Bacterial chaperones have been classified on the basis of the heat shock stress response into the Hsp100 (ClpB), Hsp70 (DnaK, in association with the Hsp40 cofactor DnaJ and the nucleotide exchange factor GrpE), Hsp60/Hsp10 (GroEL/GroES chaperonin), and sHSP (IbpA and IbpB) families. The complement of chaperones found in Archaea differs in several regards to those involved in bacterial protein folding (Laksanalamai et al. 2004). Most notable is the lack of Hsp 100 homologs in all hyperthermophile genomes examined to date. Homologs of Hsp70/DnaK are also mainly absent; instead, the analogous function is performed by prefoldin. Two classes of Hsp60 chaperonin are observed: class I complexes are most similar to GroEL/ES, while class II enzymes (sometimes referred to as thermosomes) are more closely related to the eukaryotic T-complex polypeptide 1. Finally, binding of the polypeptide chain as it emerges from the ribosome is performed by nascent polypeptide-associated complex (NAC), the functional though non-homologous equivalent of trigger factor.

The list of archaeal chaperones tested for improved folding of GFP is shown in Table 1.

TABLE 1
Archaeal chaperones used in this study

| Species | ^{T}optimum | Chaperone^{a} | Size (kDa) |
|---|---|---|---|
| Methanococcoides burtonii | 15° C. | sHSP | 17.3 |
| | | HSP60 | 58.2 |
| Methanocaldococcusjannaschii | 88° | PFD | 16.4 |
| Pyrococcusfuriosus | 100° C. | sHSP | 20.2 |
| | | PFD | 16.5 |
| | | PFD | 19.7 |
| | | HSP60 | 60.0 |
| | | NAC | 12.5 |

^{a}Functional categories of the various chaperones. sHSP, small heat shock protein; HSP60, class II chaperonin; PFD, prefoldin; NAC, nascent polypeptide-associated complex protein. Genbank accession numbers: M burtoni, ABE52862 (sHSP) (SED ID NOs. 1 AND 2, gene and protein) and ABE53016 (HSP60) (SED ID NOs. 3 AND 4, gene and protein); M. jannaschii, AAB98646 (PFD) (SED ID NOs. 5 AND 6, gene and protein); P. furiosus, AAL82007 (sHSP) (SED ID NOs. 7 AND 8, gene and protein), AAL80499 (PFD)(SED ID NOs. 9 AND 10, gene and protein), AAL80506 (PFD) (SED ID NOs. 11 AND 12, gene and protein), AAL82098 (HSP60) (SED ID NOs. 13 AND 14, gene and protein), and AAL81669 (NAC) (SED ID NOs. 15 AND 16, gene and protein).

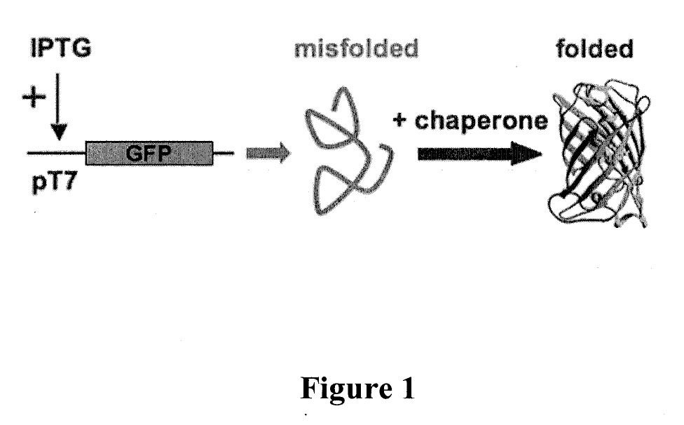

Examples were selected from both psychrophilic (low temperature) and hyperthermophilic (high temperature) species, and include prefoldins, chaperonins, sHSPs, and NAC. The genes for each of these chaperones were cloned under transcriptional control of the T7 promoter, to allow co-expression with recombinant GFP upon IPTG induction. The rationale for this approach is diagrammed in FIG. 1. Folding of GFP is extremely sensitive to temperature, and the majority of the protein accumulates as insoluble inclusion bodies when produced at 37° C. Maturation of the GFP chromophore requires proper folding of the protein (Siemering et al. 1996), so fluorescence provides a sensitive assay for chaperone-mediated folding. Enhanced folding is also predicted to increase the amount of GFP present in the soluble protein fraction. Therefore, activity of the archaeal chaperones was ascertained by increased fluorescence of GFP, and also by SDS-PAGE and staining of the total protein lysate as well as the soluble fraction.

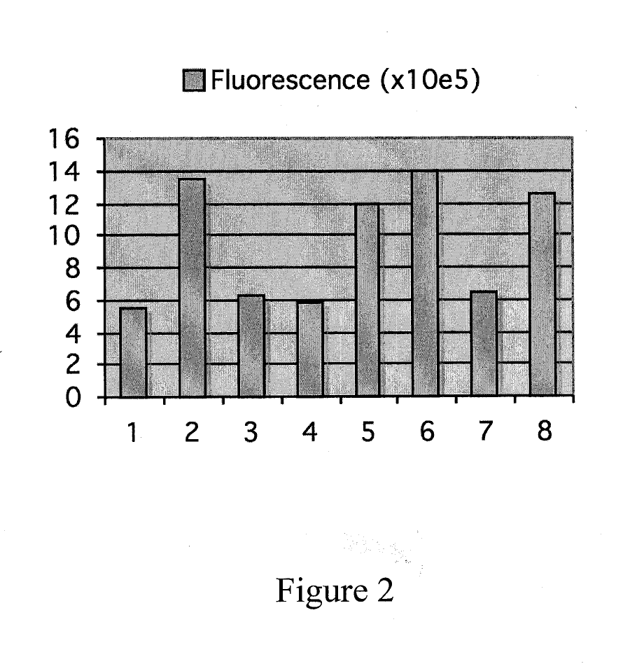

Fluorescence measurements indicate that several of the chaperones promote the proper folding of GFP under standard culture conditions for recombinant protein production (FIG. 2). In vivo activity was observed for the chaperonin and NAC from the hyperthermophile P. furiosus, the chaperonin from psychrophile M. burtonii, and prefoldin from the hyperthermophile M. jannaschii. GFP fluorescence was increased between 2 to 2.5-fold compare to cells expressing GFP alone. Three of the chaperones had no measurable effect on GFP fluorescence. Induction of one of the chaperones, sHSP from P. furiosus, caused the culture to arrest at a lower cell density and exhibit less fluorescence than the control (data not shown), suggesting that high levels of this protein might impair cell growth and GFP expression or folding.

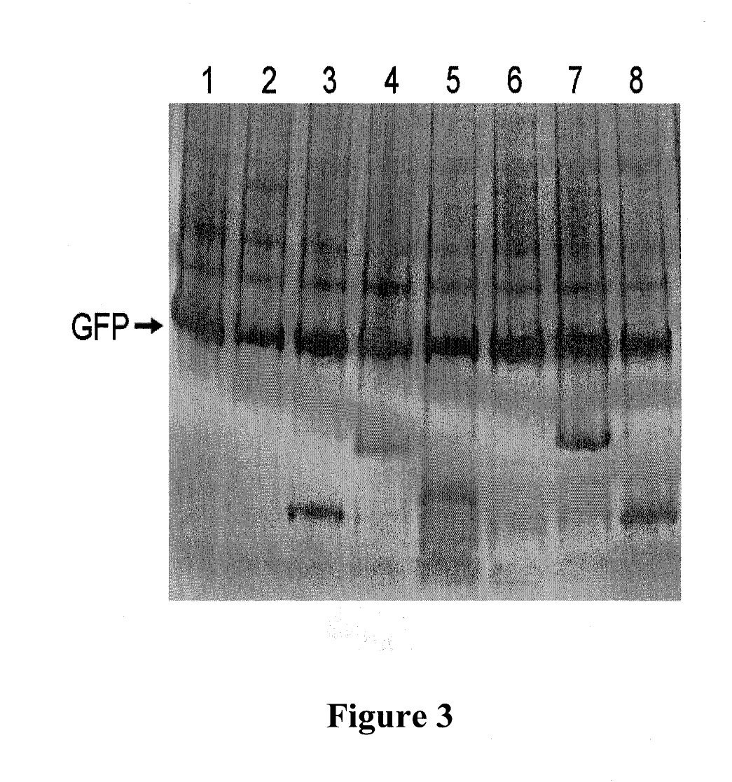

Prior characterization of the activity and solubility of an aggregation-prone GFP fusion upon co-expression of the E. coli chaperone DnaK indicated that fluorescence correlates strongly with the total amount of GFP protein but weakly or not at all with the fraction of soluble protein (Martinez-Alonso et al. 2007). If so, then increased fluorescence in the experiments might merely reflect an influence of the archaeal chaperone on GFP protein levels. However, SDS-PAGE of total cell lysates demonstrates that similar amounts of GFP are present in all samples (FIG. 3), and fluorescence measurements of fractionated protein lysates indicate that >90% of the active GFP is found in the soluble fraction (data not shown). These results demonstrate that the mechanism for enhanced GFP fluorescence by archaeal chaperones is not increased protein accumulation.

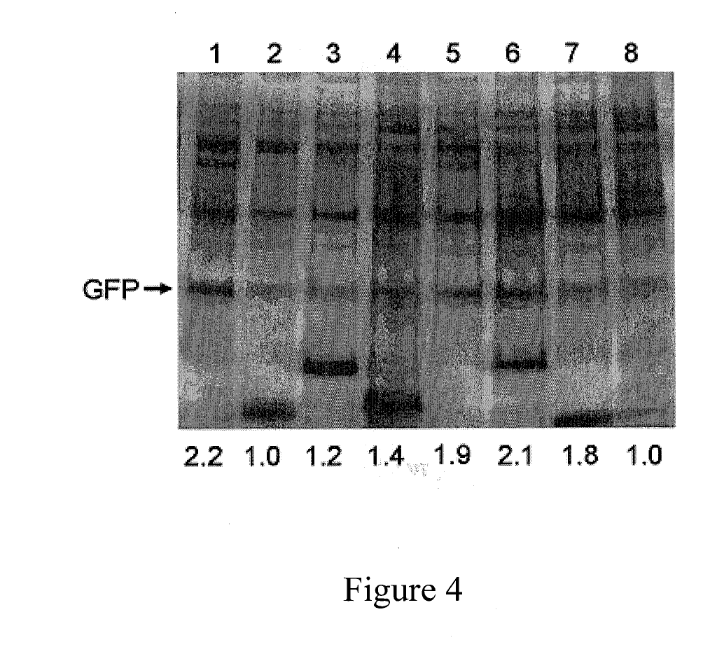

Examination of the soluble protein fractions by SDS-PAGE also indicates that some of the archaeal chaperones improve the solubility of GFP (FIG. 4). However, the increase in GFP solubility is less than the increase in fluorescence and, by itself, appears insufficient to fully explain the observed activity. Maturation of the GFP chromophore requires cyclization and oxidation of the Ser-Tyr-Gly peptide core, and this reaction is inhibited at higher temperature (Cody et al. 1993); (Heim et al. 1994); (Siemering et al. 1996). Therefore, it seems likely that some of the soluble GFP is in the apo (i.e., non-fluorescent) form. The observed increase in GFP fluorescence in the presence of archaeal chaperones appears to involve both an increase in protein solubility as well as stimulation of chromophore maturation (or resistance to inactivation).

Although the successful identification of archaeal chaperones that enhance the folding of GFP demonstrates the utility of this approach, it seems likely that further improvements can be obtained. First, it is noted that several of the chaperones are themselves partially or largely insoluble. This material is in all probability misfolded and, in addition to being inactive, is likely to compete with GFP for the limited folding capacity of the bacterial host chaperones. Engineering the system to reduce or eliminate the amount of insoluble archaeal chaperone will minimize competition for the host folding machinery. Second, induction of the archaeal chaperone occurs concurrently with induction of GFP, since each is expressed from the T7 promoter. A better strategy might involve expression of the chaperone prior to induction of the recombinant target protein, so that the chaperone is poised to facilitate folding of the newly synthesized polypeptide. Both the amount of chaperone and the timing of induction could be modulated via expression from a different inducible promoter.

Finally, archaeal chaperones from three functional classes (prefoldin, chaperonin, and NAC) were able to enhance the folding of GFP. Each of these chaperones is predicted to exhibit unique biochemical properties that promote folding by independent mechanisms, and to function at different steps in the protein-folding pathway. Therefore, combinations of archaeal chaperones likely act synergistically to improve the folding of recombinant proteins.

### REFERENCES

The contents of all references cited herein are incorporated by reference herein for all purposes.
Allen S P, Polazzi J O, Gierse J K, Easton A M. 1992. Two novel heat shock genes encoding proteins produced in response to heterologous protein expression in Escherichia coli. J Bacteriol 174(21): 6938-47.
Baneyx F, Mujacic M. 2004. Recombinant protein folding and misfolding in Escherichia coli. Nat Biotechnol 22(11):1399-408.
Chen J, Acton T B, Basu S K, Montelione G T, Inouye M. 2002 Enhancement of the solubility of proteins overexpressed in Escherichia coli by heat shock. J Mol Microbiol Biotechnol 4(6): δ 19-24.
Cody C W, Prasher D C, Westler W M, Prendergast F G, Ward W W. 1993. Chemical structure of the hexapeptide chromophore of the Aequorea green-fluorescent protein. Biochemistry 32(5): 12 12-8.
de Marco A, Deuerling E, Mogk A, Tomoyasu T, Bukau B. 2007. Chaperone-based procedure to increase yields of soluble recombinant proteins produced in E. coli. BMC Biotechnol 7:32.
Deuerling E, Schulze-Specking A, Tomoyasu T, Mogk A, Bukau B. 1999. Trigger factor and DnaK cooperate in folding of newly synthesized proteins. Nature 400(6745):693-6.
Ewalt K L, Hendrick J P, Houry W A, Hartl F U. 1997. In vivo observation of polypeptide flux through the bacterial chaperonin system. Cell 90(3):491-500.
Han M J, Park S J, Park T J, Lee S Y. 2004. Roles and applications of small heat shock proteins in the production of recombinant proteins in Escherichia coli. Biotechnol Bioeng 88(4):426-36.
Heim R, Prasher D C, Tsien R Y. 1994. Wavelength mutations and posttranslational autoxidation of green fluorescent protein. Proc Natl Acad Sci USA 91(26):12501-4.
Hoffmann F, Rinas U. 2004. Roles of heat-shock chaperones in the production of recombinant proteins in Escherichia coli. Adv Biochem Eng Biotechnol 89:143-61.
Jana S, Deb J K. 2005. Strategies for efficient production of heterologous proteins in Escherichia coli. Appl Microbiol Biotechnol 67(3):289-98.
Jenney F E, Jr., Adams M W. 2008. The impact of extremophiles on structural genomics (and vice versa). Extremophiles 12(1): 39-50.
Laksanalamai P, Maeder D L, Robb F T. 2001. Regulation and mechanism of action of the small heat shock protein from the hyperthermophilic archaeon Pyrococcus furiosus. J Bacteriol 183(17):5198-202.
Laksanalamai P, Whitehead T A, Robb F T. 2004. Minimal protein-folding systems in hyperthermophilic archaea. Nat Rev Microbiol 2(4):315-24.
Laskowska E, Wawrzynow A, Taylor A. 1996. IbpA and IbpB, the new heat-shock proteins, bind to endogenous Escherichia coli proteins aggregated intracellularly by heat shock. Biochimie 78(2):1 17-22.
Martinez-Alonso M, Vera A, Villayerde A. 2007. Role of the chaperone DnaK in protein solubility and conformational quality in inclusion body-forming Escherichia coli cells. FEMS Microbiol Lett 273(2): 187-95.
Mehlin C, Boni E, Buckner F S, Engel L, Feist T, Gelb M H, Haji L, Kim D, Liu C, Mueller N and others. 2006. Heterologous expression of proteins from Plasmodium falciparum: results from 1000 genes. Mol Biochem Parasitol 148(2):144-60.
Nishihara K, Kanemori M, Yanagi H, Yura T. 2000. Overexpression of trigger factor prevents aggregation of recombinant proteins in Escherichia coli. Appl Environ Microbiol 66(3):884-9.
Rinas U, Hoffmann F, Betiku E, Estape D, Marten S. 2007. Inclusion body anatomy and functioning of chaperone-mediated in vivo inclusion body disassembly during high-level recombinant protein production in Escherichia coli. J Biotechnol 127(2):244-57.
Siemering K R, Golbik R, Sever R, Haseloff J. 1996. Mutations that suppress the thermosensitivity of green fluorescent protein. Curr Biol 6(12):1653-63.
Sorensen H P, Mortensen K K. 2005. Soluble expression of recombinant proteins in the cytoplasm of Escherichia coli. Microb Cell Fact 4(1): 1.
Studier F W, Moffatt B A. 1986. Use of bacteriophage T7 RNA polymerase to direct selective high-level expression of cloned genes. J Mol Biol 189(1):1 13-30.
Teter S A, Houry W A, Ang D, Tradler T, Rockabrand D, Fischer G, Blum P, Georgopoulos C, Hartl F U. 1999. Polypeptide flux through bacterial Hsp70: DnaK cooperates with trigger factor in chaperoning nascent chains. Cell 97(6):755-65.
Thomas J G, Baneyx F. 1996. Protein misfolding and inclusion body formation in recombinant Escherichia coli cells overexpressing Heat-shock proteins. J Biol Chem 271(19): 11141-7.

Zolkiewski M. 1999. ClpB cooperates with DnaK, DnaJ, and GrpE in suppressing protein aggregation. A novel multi-chaperone system from Escherichia coli. J Biol Chem 274(40):28083-6.

That which is claimed is:

1. A method of enhancing protein folding in a bacteria host, the method comprising:
providing at least one expression vector comprising nucleic acid sequences encoding for a chaperone from a hyperthermophilic and/or psychrophilic archaeon and nucleic acid sequences encoding a native and/or non-native protein for expression in the host bacteria.

2. The method of claim 1, wherein the bacteria host is e coli.

3. The method of claim 1, wherein the chaperone is selected from the group consisting of prefoldin (PFD), heat shock protein, chaperonin, and nascent polypeptide-associated complex protein (NAC).

4. The method of claim 1, wherein the chaperone is expressed previously, simultaneously or subsequent to the expression of the native or non-native protein in the host.

5. The method of claim 1, wherein the chaperone is from P. furious, M. butonii or M. jannaschii.

6. The method of claim 1, wherein the chaperone is selected from the group consisting of P. furious HSP60, P. furious NAC, M. butonii HSP60 and M. jannaschii PFD.

7. A method for enhancing protein folding of a native and non-native protein in a bacteria host to provide increased level of properly folded and bioactive proteins, the methods comprising:
introducing into a bacteria host at least one expression vector comprising:
nucleic acid encoding a chaperone selected from the group consisting of prefoldin (PFD), heat shock protein, chaperonins, and/or nascent polypeptide-associated complex protein (NAC) from a hyperthermophilic and/or psychrophilic archaeon and at least one native or non-native protein; and
culturing the bacteria host under conditions sufficient for expression of the proteins and chaperones.

8. The method of claim 7, wherein the bacteria host is e coli.

9. The method of claim 7, wherein the chaperone is expressed previously, simultaneously or subsequent to the expression of the native or non-native protein in the host.

10. The method of claim 7, wherein the chaperone is from P. furious, M. butonii or M. jannaschii.

11. The method of claim 7, wherein the chaperone is selected from the group consisting of P. furious HSP60, P. furious NAC, M. butonii HSP60 and M. jannaschii PFD.

12. A method to screen for extremophilic chaperones that exhibit folding activity under bacterial growth conditions, the method comprising;
providing an expression vector comprising a nucleotide sequence that encodes for an extremophilic chaperone and an indicator protein, wherein the indicator protein provides for a detectable signal.

13. The method of claim 12, wherein the indicator protein is green fluorescence protein.

14. The method of claim 12, wherein the bacterial growth conditions is for culturing E. coli.

15. The method according to claim 12, wherein the chaperone is selected from the group consisting of P. furious HSP60, P. furious NAC, M. butonii HSP60 and M. jannaschii PFD.

16. A delivery device comprising nucleotide sequences encoding chaperones from a hyperthermophilic and/or psychrophilic archaeon, in an amount to enhance the folding of expressed native and non-native proteins in a bacteria host.

17. An assay to screen for extremophilic chaperones that exhibit folding activity under bacteria growth conditions, the method comprising;
expressing a testing extremophilic chaperone in combination with the expression of green fluorescent protein; and
determining the amount of amount of GFP recovered in the soluble protein fraction.
